(19) United States
(12) Patent Application Publication
(10) Pub. No.: US 2022/0265531 A1
(43) Pub. Date: Aug. 25, 2022

(54) ORAL CARE COMPOSITIONS COMPRISING PEROXIDE AND COMPATIBLE ABRASIVE

(71) Applicant: The Procter & Gamble Company, Cincinnati, OH (US)

(72) Inventors: Lawrence Edward Dolan, Cincinnati, OH (US); Steven Hamilton Hoke, III, West Chester, OH (US); Sanjeev Midha, Mason, OH (US); Jonathan M. Pahren, Mason, OH (US); Niranjan Ramji, Mason, OH (US); Rachel Anne Trenner, Centerville, OH (US)

(21) Appl. No.: 17/577,402

(22) Filed: Jan. 18, 2022

Related U.S. Application Data
(60) Provisional application No. 63/151,091, filed on Feb. 19, 2021.

(51) Int. Cl. A61K 8/22; A61Q 11/00; A61K 8/81; A61K 8/19; A61K 8/24; A61K 8/44; A61K 8/49; A61K 8/21; A61K 8/27; A61K 8/46

(52) U.S. Cl. CPC A61K 8/22; A61Q 11/00; A61K 8/8188; A61K 8/19; A61K 8/24; A61K 8/44; A61K 8/4946; A61K 8/492; A61K 8/21; A61K 8/27; A61K 8/463; A61K 2800/5428; A61K 2800/28

(57) ABSTRACT

Oral care compositions including peroxide and abrasive. Dentifrice compositions including peroxide and abrasive. Oral care compositions including peroxide and calcium pyrophosphate. Dentifrice compositions including peroxide and calcium pyrophosphate. Dentifrice compositions including peroxide and calcium pyrophosphate with a gamma phase to beta phase ratio of at least about 0.8. Dentifrice composition including peroxide and calcium pyrophosphate with a gamma phase to beta phase ratio of at least about 1. Methods of use of the disclosed compositions to whiten teeth.

### FIELD OF THE INVENTION

The present invention relates to oral care compositions comprising peroxide and abrasive. The present invention relates to dentifrice compositions comprising peroxide and peroxide-compatible abrasive. The present invention also relates to dentifrice compositions comprising peroxide and calcium pyrophosphate. The present invention also relates to dentifrice compositions comprising peroxide and calcium pyrophosphate with a ratio of gamma phase calcium pyrophosphate to beta phase calcium pyrophosphate of at least about 0.8. The present invention also related to dentifrice compositions comprising peroxide and abrasive, wherein the abrasive comprises less than about 0.0006%, by weight of the oral care composition, of soluble metal selected from the group consisting of Cr, Mn, Fe, Co, Ni, Cu, and combinations thereof.

### BACKGROUND OF THE INVENTION

Oral care compositions, such as dentifrice compositions, can include fluoride, peroxide, and/or abrasive to clean teeth, prevent cavities, and maintain the aesthetics and health of the oral cavity, such as the teeth and gums.

It can be desirable to have dentifrice compositions with a relatively high level of peroxide, such as at least about 1%, at least about 3%, or at least about 3.5%, to achieve enhanced whitening efficacy. However, it can be challenging to formulate dentifrice compositions with a relatively high level of peroxide because peroxide can be reactive with other dentifrice components, such as fluoride, metal ions, abrasives, etc. One approach to the formulation challenges associated with peroxide can be to separately package and/or apply peroxide from a separate whitening composition. However, consumer compliance can be lower when peroxide must be applied from a separate composition. Thus, it can be advantageous to provide peroxide in a dentifrice composition with other components, such as fluoride.

When peroxide decomposes, it forms oxygen, water, and radicals. The excess gas can cause swelling and bursting of primary packaging and the radicals can interact with other dentifrice components, such as fluoride, metal ions, abrasives, etc. In particular, many abrasives can be reactive with peroxide. As such, there is a need for an improved oral care composition, such as an improved dentifrice composition, including peroxide and abrasive.

### SUMMARY OF THE INVENTION

Disclosed herein is an oral care composition comprising (a) peroxide; (b) calcium pyrophosphate; and (c) water, wherein the oral care composition comprises a ratio of gamma phase calcium pyrophosphate to beta phase calcium pyrophosphate of at least about 0.8.

Also disclosed herein is an oral care composition comprising (a) peroxide; (b) abrasive; and (c) water, wherein the abrasive comprises less than about 0.001%, by weight of the oral care composition, of metal.

Also disclosed herein is an oral care composition comprising (a) peroxide; (b) calcium pyrophosphate; and (c) water, wherein the oral care composition has at least about 90% of peroxide remaining after the oral care composition has been stored at 50° C. for 10 days.

Also disclosed herein is an oral care composition comprising (a) peroxide; (b) calcium pyrophosphate; and (c) water, wherein the calcium pyrophosphate has a slurry pH of from about 4 to about 7.

Also disclosed herein is an oral care composition comprising (a) peroxide; (b) abrasive; and (c) water, wherein the abrasive comprises less than about 0.0006%, by weight of the oral care composition, of soluble metal selected from the group consisting of Cr, Mn, Fe, Co, Ni, Cu, and combinations thereof.

Also disclosed herein is an oral care composition comprising (a) peroxide; (b) calcium pyrophosphate; and (c) water, wherein the calcium pyrophosphate comprises at least one of: (i) a ratio of gamma phase calcium pyrophosphate to beta phase calcium pyrophosphate of at least about 0.8; (ii) a slurry pH of from about 5.4 to about 7; or (iii) less than about 0.0006%, by weight of the calcium pyrophosphate, of soluble metal selected from the group consisting of Cr, Mn, Fe, Co, Ni, Cu, and combinations thereof.

### DETAILED DESCRIPTION OF THE INVENTION

The present invention is directed to oral care compositions comprising peroxide and a peroxide-compatible abrasive. While not wishing to being bound by theory, it is believed that peroxide can interact with the surface of many abrasives and/or trace amounts of metal ions in many abrasives.

It has been surprisingly found that certain abrasives can be compatible with peroxide through the reduction in the amount of soluble metal ions and/or designing the abrasive to have a higher amount of certain polymorphs.

Calcium pyrophosphate is a commonly used abrasive in dentifrice compositions. Calcium pyrophosphate has at least three polymorphs: alpha (α), beta (β), and gamma (γ). It has been surprisingly found that calcium pyrophosphate with a higher amount of the gamma polymorph are more compatible with peroxide. Thus, the present invention is directed to oral care compositions with peroxide and peroxide-compatible abrasive, such as calcium pyrophosphate with a high amount of gamma polymorph and/or a certain ratio of gamma polymorph to beta polymorph.

It has also been surprisingly found that certain abrasives can be compatible with peroxide through the reduction in the amount of soluble metal ions. While not wishing to being bound by theory, it is believed that soluble metal ions, such as Cr, Mn, Fe, Co, Ni, Cu, or combinations thereof, can act as reducing agents to interact with peroxide, leading to the degradation of peroxide. Thus, the present invention is directed to oral care compositions with peroxide and peroxide-compatible abrasive, such as abrasive with a total metal content of less than about 0.0006%, by weight of the oral care composition of soluble metal selected from the group consisting of Cr, Mn, Fe, Co, Ni, Cu, and combinations thereof.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

The term “oral hygiene regimen” or “regimen” can be for the use of two or more separate and distinct treatment steps for oral health. e.g. toothpaste, mouth rinse, floss, toothpicks, spray, water irrigator, massager.

The term “total water content” as used herein means both free water and water that is bound by other ingredients in the oral care composition.

For the purpose of the present invention, the relevant molecular weight (MW) to be used is that of the material added when preparing the composition e.g., if the chelant is a citrate species, which can be supplied as citric acid, sodium citrate or indeed other salt forms, the MW used is that of the particular salt or acid added to the composition but ignoring any water of crystallization that may be present.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The dentifrice composition can be in any suitable form, such as a solid, liquid, powder, paste, or combinations thereof. The oral care composition can be dentifrice, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the dentifrice composition can be incorporated into a film, a strip, a foam, or a fiber-based dentifrice composition.

The oral care compositions, as described herein, comprise peroxide and peroxide compatible abrasive, such as calcium pyrophosphate. Additionally, the oral care compositions can comprise other optional ingredients, as described below. The section headers below are provided for convenience only. In some cases, a compound can fall within one or more sections. For example, stannous fluoride can be a tin compound and/or a fluoride compound.

### Peroxide

The oral care composition comprises peroxide. The peroxide can include any suitable source of peroxide, such as solubilized peroxide compounds and/or solid peroxide sources. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, polyvinylpyrrolidone-peroxide complex, crosslinked polyvinylpyrrolidone-peroxide complex, and/or combinations thereof.

While not wishing to being bound by theory, it is believed that peroxide compounds that are stabilized through the complexation with a stabilizing compound, such as crosslinked polyvinyl pyrrolidone-peroxide, are less available to provide an oral health benefit, such as a whitening benefit, when applied to the oral cavity. Thus, the peroxide can comprise solubilized peroxide compounds, such as aqueous hydrogen peroxide.

Additionally, the oral care composition can be designed, such as through the selection of the other components of the oral care composition, to maximize the amount of remaining peroxide after a defined period after initially mixing the oral care composition. Suitable oral care compositions include oral care compositions comprising at least about 75%, at least about 80%, at least about 85%, at least about 90%, from about 90% to about 99%, or from about 88% to about 99% of hydrogen peroxide remaining in the oral care composition the oral care composition is stored at 50° C. for 10 days. Other suitable oral care compositions include oral care compositions comprising at least about 75%, at least about 80%, at least about 85%, at least about 90%, at least about 95%, from about 90% to about 99%, or from about 88% to about 99% of hydrogen peroxide remaining in the oral care composition the oral care composition is stored at 50° C. for 20 days.

The oral care composition can comprise from about 0.01% to about 20%, from about 0.01% to about 10%, from about 1% to about 5%, at least about 1%, at least about 2%, at least about 3%, at least about 3.5%, at least about 4%, or greater than 3%, by weight of the oral care composition, of the peroxide.

While not wishing to being bound by theory, it is believed that the peroxide, as described herein, can be un-bound, uncomplexed, and/or only minimally stabilized by other components in the oral care composition. It is additionally believed that oral care compositions comprising peroxide that is un-bound, uncomplexed, and/or only minimally stabilized to other components in the oral care composition, such as amphiphilic polymer and/or other thickening agents, can lead to a greater rate of release of peroxide than compositions that have peroxide adducts, such as polyvinylpyrrolidone-peroxide, and/or other sources of bound peroxide. Desirable oral care compositions include oral care compositions that have a peroxide release of at least about 10%, at least about 15%, at least about 25%, at least about 35%, at least about 50%, at least about 60%, at least about 70%, at least about 75%, at least about 80%, at least about 90%, at least about 95%, from about 10% to about 95%, from about 25% to about 75%, from about 20% to about 95%, from about 35% to about 90%, from about 40% to about 95%, from about 50% to about 90%, and from about 60% to about 99% in a defined period of time corresponding to a single oral care session, such as 30 second, 45 second, 60 seconds, 75 seconds, 90 seconds, 120 seconds, and/or 5 minutes.

### Abrasive

The oral care composition comprises abrasive, such as a peroxide-compatible abrasive. Abrasives can be added to oral care formulations to help remove surface stains from teeth. The abrasive can comprise calcium abrasive, silica abrasive, carbonate abrasive, phosphate abrasive, alumina abrasive, other suitable abrasives, and/or combinations thereof.

The calcium abrasive can comprise calcium carbonate, dicalcium phosphate, tricalcium phosphate, calcium orthophosphate, calcium metaphosphate, calcium pyrophosphate, calcium polyphosphate, calcium hydroxyapatite, and combinations thereof.

The calcium abrasive can comprise calcium carbonate. The calcium-containing abrasive can be selected from the group consisting of fine ground natural chalk, ground calcium carbonate, precipitated calcium carbonate, and combinations thereof.

The calcium abrasive can comprise calcium pyrophosphate. While not wishing to be bound by theory, it is believed that particular sources of calcium pyrophosphate can be sufficiently compatible with peroxide to be used in an oral care composition comprising peroxide. Calcium pyrophosphate has at least three polymorphic phases: alpha (α), beta (β), and gamma (γ). It has been unexpectedly found that calcium pyrophosphate with a higher proportion of the γ phase are more compatible with peroxide, such as hydrogen peroxide. Suitable sources of calcium pyrophosphate can comprise a ratio of gamma phase calcium pyrophosphate to beta phase calcium pyrophosphate of at least about 0.75, at least about 0.8. at least about 0.9, at least about 1, from about 0.75 to about 1.5, from about 0.9 to about 1.5, or from about 1 to about 1.5.

While not wishing to being bound by theory, it is believed that abrasives that include high amounts of soluble metal ions are less compatible with peroxide due to reactivity between peroxide and metal ions. Thus, suitable sources of abrasives, such as calcium pyrophosphate, can comprise less than about 0.001%, less than about 0.00075%, less than about 0.0006%, less than about 0.0005%, or less than about 0.0001%, by weight of the abrasive. Additionally, the abrasive can be free of, substantially free of, or essentially free of soluble metal ions. Examples of trace metal ions that might be present in abrasives and have been previously reported to induce peroxide degradation include Cr, Mn, Fe, Co, Ni, Cu, Mo, and/or combinations thereof.

As described herein, it has also been unexpectedly found that abrasives with a slurry pH of from about 5.4 to about 7 were more compatible with peroxide. The slurry pH was determined by creating a 1:3 abrasive:water slurry and determining the pH.

The carbonate abrasive can comprise sodium carbonate, sodium bicarbonate, calcium carbonate, strontium carbonate, and/or combinations thereof.

The phosphate abrasive can comprise calcium phosphate, sodium hexametaphosphate, dicalcium phosphate, tricalcium phosphate, calcium orthophosphate, calcium metaphosphate, calcium polyphosphate, a polyphosphate, a pyrophosphate, and/or combinations thereof.

The silica abrasive can comprise fused silica, fumed silica, precipitated silica, hydrated silica, and/or combinations thereof.

The alumina abrasive can comprise polycrystalline alumina, calcined alumina, uncalcined alumina, fused alumina, levigated alumina, hydrated alumina, and/or combinations thereof.

Other suitable abrasives include diatomaceous earth, barium sulfate, wollastonite, perlite, polymethylmethacrylate particles, tospearl, and combinations thereof.

The oral care composition can comprise from about 0.01% to about 30%, from about 0.01% to about 15%, from about 0.01% to less than 15%, from about 1% to about 30%, from about 1% to less than 15%, from about 1% to about 12%, or from about 0.01% to about 10%, by weight of the oral care composition of the abrasive.

The oral care composition can have a Pellicle Cleaning Ratio (PCR) of at least about 75, at least about 90, at least about 100, from about 75 to about 250, or from about 100 to about 250.

The oral care composition can have a Relative Dentin Abrasion value of up to about 250, up to about 150, from about 70 to about 150, or from about 50 to about 250.

### Alumina

The oral care composition can include alumina as an abrasive. The alumina can be the sole abrasive or used in combination with other abrasives, as described herein. The abrasivity of alumina can be higher than silica, thus a lower amount of alumina abrasive can be added.

An oral care composition comprising alumina can have a hard tissue safety value (REA) of at least about 15, at least about 20, at least about 25, or at least about 40.

An oral care composition comprising alumina can have a pellicle cleaning ratio (PCR) of at least about 75, at least about 100, from about 75 to about 250, from about 50 to about 175, from about 50 to about 150, or rom about 65 to about 155.

An oral care composition comprising alumina can have a Relative Dentin Abrasion (RDA) of less than about 150, less than about 125, less than about 100, or less than about 90.

While not wishing to be bound by theory, it is believed that alumina can be reactive with peroxide and/or fluoride. Thus, desirable alumina abrasives include those that are compatible with peroxide and/or fluoride. Suitable oral care compositions include oral care compositions comprising at least about 75%, at least about 80%, at least about 85%, at least about 90%, at least about 95%, from about 90% to about 99%, or from about 88% to about 99% of hydrogen peroxide remaining in the oral care composition the oral care composition is stored at 40 ° C. for 30 and/or 60 days.

Other suitable oral care compositions include oral care compositions that comprise peroxide and alumina and have % loss of peroxide after 30 days and/or 90 days at 40 ° C. of less than about 10%, less than about 5%, less than about 4%, less than about 3%, from about 0.5% to about 10%, from about 0.5% to about 5%, or from about 0.1% to about 5%. While not wishing to being bound by theory, it is believed that alumina is unexpectedly stable with alumina, such that only a minimal amount of peroxide is lost to peroxide decomposition and/or degradation.

Other suitable oral care compositions include oral care compositions that comprise peroxide, fluoride, and alumina and have % loss of fluoride after 30 days and/or 90 days at 40 ° C. of less than about 20%, less than about 18%, less than about 15%, less than about 10%, less than about 5%, from about 0.5% to about 10%, from about 0.5% to about 20%, or from about 0.1% to about 15%. While not wishing to being bound by theory, it is believed that alumina and peroxide are unexpectedly stable with certain fluoride sources, such that only a minimal loss of fluoride is observed.

Other suitable oral care compositions include oral care compositions comprising peroxide and alumina and have a % increase of viscosity 30 days and/or 90 days at 40 ° C. of at least about 5%, at least about 10%, at least about 15%, at least about 20%, at least about 25%, at least about 35%, at least about 50%, at least about 75%, at least about 100%, from about 5% to about 125%, from about 5% to about 50%, or from about 1%, to about 75%. While not wishing to being bound by theory, it is believed that the combination of alumina and peroxide are unexpectedly stable, which can minimize the decrease in viscosity in meta-stable peroxide compositions normally observed due to the decomposition of peroxide.

The oral care composition can comprise from about 0.01% to about 10%, from about 0.01% to about 5%, from about 0.1% to about 5%, from about 0.01% to about 3%, or from about 0.01% to about 1%, by weight of the oral care composition, of alumina.

### Water

The oral care composition of the present invention can be a dentifrice composition that is anhydrous, a low water formulation, or a high water formulation. In total, the oral care composition can comprise from 0% to about 99%, from about 5% to about 60%, from about 5% to about 75%, about 20% or greater, about 30% or greater, about 50% or greater, up to about 45%, or up to about 75%, by weight of the composition, of water. Preferably, the water is USP water.

The oral care composition of the present invention can include water. While not wishing to be bound by theory, it is believed that including water in the oral care composition can lead to greater availability of peroxide to provide oral health benefits, such as whitening, while also hydrating the oral cavity. While one option to stabilize peroxide is to provide a complexed peroxide in an anhydrous dentifrice composition, it is believed that peroxide can be more effectively delivered through the selection of the other ingredients of the oral care composition while simultaneously hydrating the oral cavity.

In a high water dentifrice formulation, the dentifrice composition comprises from about 45% to about 75%, by weight of the composition, of water. The high water dentifrice composition can comprise from about 45% to about 65%, from about 45% to about 55%, or from about 46% to about 54%, by weight of the composition, of water. The water may be added to the high water dentifrice formulation and/or may come into the composition from the inclusion of other ingredients.

In a low water dentifrice formulation, the dentifrice composition comprises from about 5% to about 45% or from about 10% to about 45%, by weight of the composition, of water. The low water dentifrice composition can comprise from about 10% to about 35%, from about 15% to about 25%, or from about 20% to about 25%, by weight of the composition, of water. The water may be added to the low water dentifrice formulation and/or may come into the composition from the inclusion of other ingredients.

In an anhydrous dentifrice formulation, the dentifrice composition comprises less than about 10%, by weight of the composition, of water. The anhydrous dentifrice composition comprises less than about 5%, less than about 1%, or 0%, by weight of the composition, of water. The water may be added to the anhydrous formulation and/or may come into the dentifrice composition from the inclusion of other ingredients.

The dentifrice composition can also comprise other orally acceptable carrier materials, such as alcohol, humectants, polymers, surfactants, and acceptance improving agents, such as flavoring, sweetening, coloring and/or cooling agents.

The oral care composition can also be a mouth rinse formulation. A mouth rinse formulation can comprise from about 75% to about 99%, from about 75% to about 95%, or from about 80% to about 95% of water.

### Amphiphilic Polymer

The oral care composition can comprise amphiphilic polymer. The amphiphilic polymer can be included as a peroxide compatible thickening agent. The amphiphilic polymer can comprise a polymer that has a hydrophobic portion and a hydrophilic portion. For example, the polymer can comprise a polymer macromolecule that comprises a hydrophilic backbone and hydrophobic subunits. This can allow the amphiphilic polymer to remain soluble in water while preventing strong interactions with the peroxide.

As described herein, it is believed that peroxide can be effectively delivered in an aqueous chassis if the other components are designed to minimize reactivity with the peroxide. As such, it is desirable for the amphiphilic polymer to not strongly react with the peroxide. Thus, it is desirable for the amphiphilic polymer to not form an isolatable complex with the peroxide.

The amphiphilic polymer can include polymers that are at least partially soluble and/or fully soluble in water.

Suitable amphiphilic polymers include 2-acrylamido-2-methylpropane sulfonic acid (AMPS) polymer, copolymer, cross-polymer, or combination thereof. In one example, the oral care composition can contain polyacrylate crosspolymer-6 (commercially available as SepiMAX™ ZEN from SEPPIC S.A., a subsidiary of the Air Liquide group, Puteaux Cedex, France).

The oral care composition can comprise from about 0.01% to about 10%, from about 0.1% to about 5%, from about 1% to about 10%, or from about 1% to about 5%, by weight of the oral care composition, of the amphiphilic polymer.

### Alkyl Alcohol

The oral care composition can comprise alkyl alcohol. While not wishing to be bound by theory, it is believed that the addition of alkyl alcohol in combination with the amphiphilic polymer can lead to oral care compositions with unexpectedly high viscosities.

The alkyl alcohol can include compounds with an alkyl functional group and an alcohol functional group. The alkyl functional can be linear, branched, cyclical, or combinations thereof. The alkyl alcohol can include a primary alcohol, a secondary alcohol, and/or a tertiary alcohol.

The alkyl alcohol can be represented by the general formula of CₙH₂ₙ₊₁OH, wherein n can be any whole number from 1 to 30, from 10 to 20, or from 10 to 25.

Specific examples of alkyl alcohols can include 1-heptacosanol, 1-hexacosanol, 1-nonacosanol, 1-octacosanol, 1-tetracosanol, docosanol, heneicosan-1-ol, pentacosan-1-ol, tricosan-1-ol, 1,4-butanediol, 1-heptanol, 1-hexanol, 1-nonanol, 1-octanol, 1-pentanol, 1-propanol, 2,4-dichlorobenzyl alcohol, 2-ethylhexanol, 3-nitrobenzyl alcohol, allyl alcohol, anisyl alcohol, arachidyl alcohol, benzyl alcohol, cetyl alcohol, cinnamyl alcohol, crotyl alcohol, furfuryl alcohol, isoamyl alcohol, neopentyl alcohol, nicotinyl alcohol, perillyl alcohol, phenethyl alcohol, propargyl alcohol, salicyl alcohol, stearyl alcohol, tryptophol, vanillyl alcohol, veratrole alcohol, and/or combinations thereof. The alkyl alcohol can comprise stearyl alcohol, cetyl alcohol, and/or combinations thereof.

The alkyl alcohol can be provided through commercial mixtures of alkyl alcohols, such as Lanette® W, which includes cetyl alcohol:stearyl alcohol:sodium lauryl sulfate at a 45:45:10 ratio (available from BASF, Florham Park, N.J.).

The oral care composition can comprise from about 0.01% to about 10%, from about 0.1% to about 5%, from about 1% to about 10%, or from about 1% to about 5%, by weight of the oral care composition, of the alkyl alcohol.

The combination of amphiphilic polymer and alkyl alcohol can lead to oral care compositions with unexpectedly high viscosities. Suitable ratios of amphiphilic polymer:alkyl alcohol can be at least about 0.25, at least about 0.5, at least about 0.75, at least about 1, from about 0.25 to about 5, about 0.5 to 1.5, or about 1.

The oral care composition can have a viscosity of at least about 5 cP, at least about 10 cP, at least about 20 cP, from about 5 cP to about 20 cP, or from about 1 cP to about 25 cP.

pH

The pH of the disclosed composition can be from about 4 to about 10, from about 4 to about 7, from about 4 to about 8, or from about 7 to about 10.

### Fluoride

The oral care composition can comprise fluoride, which can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The fluoride ion source can be essentially free of, or free of stannous fluoride. Thus, the oral care composition can comprise sodium fluoride, potassium fluoride, amine fluoride, sodium monofluorophosphate, zinc fluoride, and/or mixtures thereof.

The oral care composition can comprise a fluoride ion source capable of providing from about 50 ppm to about 5000 ppm, and preferably from about 500 ppm to about 3000 ppm of free fluoride ions. To deliver the desired amount of fluoride ions, the fluoride ion source may be present in the oral care composition at an amount of from about 0.0025% to about 5%, from about 0.01% to about 10%, from about 0.2% to about 1%, from about 0.5% to about 1.5%, or from about 0.3% to about 0.6%, by weight of the oral care composition. Alternatively, the oral care composition can comprise less than 0.1%, less than 0.01%, be essentially free of, be substantially free of, or free of a fluoride ion source.

### Metal

The oral care composition, as described herein, can comprise metal, which can be provided by a metal ion source comprising one or more metal ions. The metal ion source can comprise or be in addition to the tin ion source and/or the zinc ion source, as described herein. Suitable metal ion sources include compounds with metal ions, such as, but not limited to Sn, Zn, Cu, Mn, Mg, Sr, Ti, Fe, Mo, B, Ba, Ce, Al, In and/or mixtures thereof. The metal ion source can be any compound with a suitable metal and any accompanying ligands and/or anions.

Suitable ligands and/or anions that can be paired with metal ion sources include, but are not limited to acetate, ammonium sulfate, benzoate, bromide, borate, carbonate, chloride, citrate, gluconate, glycerophosphate, hydroxide, iodide, oxalate, oxide, propionate, D-lactate, DL-lactate, orthophosphate, pyrophosphate, sulfate, nitrate, tartrate, and/or mixtures thereof.

The oral care composition can comprise from about 0.01% to about 10%, from about 1% to about 5%, or from about 0.5% to about 15% of metal and/or a metal ion source.

### Tin

The oral care composition of the present invention can comprise tin, which can be provided by a tin ion source. The tin ion source can be any suitable compound that can provide tin ions in an oral care composition and/or deliver tin ions to the oral cavity when the oral care composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Tin ion source can comprise stannous fluoride, stannous chloride, and/or mixture thereof. The tin ion source can also be a fluoride-free tin ion source, such as stannous chloride.

The oral care composition can comprise from about 0.0025% to about 5%, from about 0.01% to about 10%, from about 0.2% to about 1%, from about 0.4% to about 1%, or from about 0.3% to about 0.6%, by weight of the oral care composition, of tin and/or a tin ion source. Alternatively, the oral care composition can be essentially free of, substantially free of, or free of tin.

### Zinc

The oral care composition can comprise zinc, which can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate. The zinc ion source can be a fluoride-free zinc ion source, such as zinc phosphate, zinc oxide, and/or zinc citrate.

The zinc and/or zinc ion source may be present in the total oral care composition at an amount of from about 0.01% to about 10%, from about 0.2% to about 1%, from about 0.4% to about 1%, or from about 0.3% to about 0.6%, by weight of the dentifrice composition. Alternatively, the oral care composition can be essentially free of, substantially free of, or free of zinc.

### Polyphosphate

The oral care composition can comprise polyphosphate, which can be provided by a polyphosphate source. A polyphosphate source can comprise one or more polyphosphate molecules.

Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates are those having an average of two or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 2 to about 21. Alkali earth metal cations, such as calcium, are not preferred because they tend to form insoluble fluoride salts from aqueous solutions comprising a fluoride ions and alkali earth metal cations. Thus, the oral care compositions disclosed herein can be free of or substantially free of calcium pyrophosphate.

Some examples of suitable polyphosphate molecules include, for example, pyrophosphate (n=2), tripolyphosphate (n=3), tetrapolyphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 2070, or about 10% or less, by weight of the oral care composition, of the polyphosphate source. Alternatively, the oral care composition can be essentially free of, substantially free of, or free of polyphosphate,

### Orthophosphate

The oral care composition can comprise orthophosphate, which can be provided by an orthophosphate source. An orthophosphate source can comprise a salt including the orthophosphate anion, a salt including a phosphate anion (H₂PO₄²⁻, HPO₄²⁻, and PO₄³⁻), a phosphoric acid compound, a polyphosphate source, which can breakdown into orthophosphate under a variety of conditions, and/or another suitable orthophosphate source.

The oral care composition can comprise from about 0.01% to about 15%, from about 0.1% to about 10%, from about 0.5% to about 5%, from about 1 to about 20%, or about 10% or less, by weight of the oral care composition, of the orthophosphate. Alternatively, the oral care composition can be essentially free of, substantially free of, or free of orthophosphate.

### Surfactant

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

It was surprisingly found that additional amounts of anionic surfactant, such as alkyl sulfate surfactant and/or sodium lauryl sulfate, can lead to increased peroxide stability. As such, desirable compositions include compositions that comprise at least about 1.5%, greater than 1.5%, from about 1.5% to about 10%, or greater than 1.5% to about 10%, by weight of the oral care composition, of anionic surfactant.

### Amino Acid

The oral care composition can comprise amino acid. The amino acid can comprise one or more amino acids, peptide, and/or polypeptide, as described herein.

Amino acids, as in Formula II, are organic compounds that contain an amine functional group, a carboxyl functional group, and a side chain (R in Formula II) specific to each amino acid. Suitable amino acids include, for example, amino acids with a positive or negative side chain, amino acids with an acidic or basic side chain, amino acids with polar uncharged side chains, amino acids with hydrophobic side chains, and/or combinations thereof. Suitable amino acids also include, for example, arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, and/or combinations thereof.

Suitable amino acids include the compounds described by Formula I, either naturally occurring or synthetically derived. The amino acid can be zwitterionic, neutral, positively charged, or negatively charged based on the R group and the environment. The charge of the amino acid, and whether particular functional groups, can interact with tin at particular pH conditions, would be well known to one of ordinary skill in the art.

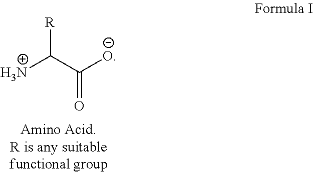

Suitable amino acids include one or more basic amino acids, one or more acidic amino acids, one or more neutral amino acids, or combinations thereof.

The oral care composition can comprise from about 0.01% to about 20%, from about 0.1% to about 10%, from about 0.5% to about 6%, or from about 1% to about 10% of amino acid, by weight of the oral care composition.

The term “neutral amino acids” as used herein include not only naturally occurring neutral amino acids, such as alanine, asparagine, cysteine, glutamine, glycine, isoleucine, leucine, methionine, phenylalanine, proline, serine, threonine, tryptophan, tyrosine, valine, but also biologically acceptable amino acids which have an isoelectric point in range of pH 5.0 to 7.0. The biologically preferred acceptable neutral amino acid has a single amino group and carboxyl group in the molecule or a functional derivative hereof, such as functional derivatives having an altered side chain albeit similar or substantially similar physio chemical properties. In a further embodiment the amino acid would be at minimum partially water soluble and provide a pH of less than 7 in an aqueous solution of 1 g/1000 ml at 25° C.

Accordingly, neutral amino acids suitable for use in the invention include, but are not limited to, alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, salts thereof, or mixtures thereof. Preferably, neutral amino acids used in the composition of the present invention may include asparagine, glutamine, glycine, salts thereof, or mixtures thereof. The neutral amino acids may have an isoelectric point of 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0, in an aqueous solution at 25° C. Preferably, the neutral amino acid is selected from proline, glutamine, or glycine, more preferably in its free form (i.e. uncomplexed). If the neutral amino acid is in its salt form, suitable salts include salts known in the art to be pharmaceutically acceptable salts considered to be physiologically acceptable in the amounts and concentrations provided.

### Humectant

The oral care composition can comprise one or more humectants, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Thickening Agents

The oral care composition can comprise one or more thickening agents in addition to the amphiphilic polymer and alkyl alcohol, as described herein. Thickening agents can be useful in the oral care compositions to provide a gelatinous structure that stabilizes the dentifrice and/or toothpaste against phase separation. Suitable thickening agents include polysaccharides, polymers, and/or silica thickeners.

The thickening agent can comprise one or more polysaccharides. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, microcrystalline cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

Other polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The thickening agent can comprise one or more polymers. The polymer can be a polyethylene glycol (PEG), a polyvinylpyrrolidone (PVP), polyacrylic acid, a polymer derived from at least one acrylic acid monomer, a copolymer of maleic anhydride and methyl vinyl ether, a crosslinked polyacrylic acid polymer, of various weight percentages of the oral care composition as well as various ranges of average molecular ranges. Alternatively, the oral care composition can be free of, essentially free of, or substantially free of a copolymer of maleic anhydride and methyl vinyl ether.

The thickening agent can comprise one or more inorganic thickening agents. Some non-limiting examples of suitable inorganic thickening agents include colloidal magnesium aluminum silicate, silica thickeners. Useful silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas.

The oral care composition can comprise from 0.01% to about 15%, from 0.1% to about 10%, from about 0.2% to about 5%, or from about 0.5% to about 2% of one or more thickening agents.

### Dicarboxylic Acid

The oral care composition can comprise dicarboxylic acid. The dicarboxylic acid comprises a compound with two carboxylic acid functional groups. The dicarboxylic acid can comprise a compound or salt thereof defined by Formula II.

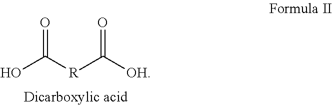

R can be null, alkyl, alkenyl, allyl, phenyl, benzyl, aliphatic, aromatic, polyethylene glycol, polymer, O, N, P, or combinations thereof.

The dicarboxylic acid can comprise oxalic acid, malonic acid, succinic acid, glutaric acid, adipic acid, pimelic acid, suberic acid, azerlaic acid, sebacic acid, undecanedioic acid, dodecanedioic acid, brassylic acid, thapsic acid, japanic acid, phellogenic acid, equisetolic acid, malic acid, tartaric acid, salts thereof, or combinations thereof. The dicarboxylic acid can comprise suitable salts of dicarboxylic acid, such as, for example, monoalkali metal oxalate, dialkali metal oxalate, monopotassium monohydrogen oxalate, dipotassium oxalate, monosodium monohydrogen oxalate, disodium oxalate, titanium oxalate, and/or other metal salts of oxalate. The dicarboxylic acid can also include hydrates of the dicarboxylic acid and/or a hydrate of a salt of the dicarboxylic acid.

The oral care composition can comprise from about 0.01% to about 10%, from about 0.1% to about 15%, from about 1% to about 5%, or from about 0.0001 to about 25%, of dicarboxylic acid.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, other caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### EXAMPLES

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### X-Ray Diffraction

Samples were prepared for X-ray diffraction by placing a sample of alumina in TABLE 1 into a mortar. The samples were pressed by applying vertical force to the sample with a pestle. Care was taken to prevent rubbing on the surface of the sample. Instead, samples were only pressed with the pestle. Samples were loaded into a sample holder and pressed with a microscope slide. The sample holder was then loaded into a TTK 450 (Anton Paar, Graz, Austria) stage.

The samples were run on a Bruker D8 Advance XRD (Billerica, Mass.) at an angle of 25-34° with 528 steps and with a time/step of 2.5 This led to a total scan time of about 31 minutes. The scan axis was 2 Theta Theta with a default scan voltage of 40 kV and a default scan current of 30 mA.

Samples were analyzed using the MDI JadePRO v.7.8.2 software. The analyzed range was 25-34° 20 with a Pearson VII function using a linear background, unify skew, and initialize and fit programming.

### Slurry pH

A slurry (6 g of Calcium Pyrophosphate and 18 g of deionized water) was made in a specimen cup. The solution was stirred for 10 minutes using a stir bar, and then the sample was centrifuged for 10 minutes at 15,000 rcf. The supernatant was poured off into a separate analysis container and pH was obtained.

### Elemental Analysis of Abrasive Slurry

### Preparation of Calcium Pyrophosphate Extracts;

0.5 g of calcium pyrophosphate was stirred with 5 g of Nitrogen (N₂) purged phosphate buffer (20 mM, pH 7.4) for 30 minutes with magnetic stirrer in a sample cup with a lid. The sample was then decanted into a Eppendorf tube and centrifuged at 5000 rpm for 30 minutes. The supernatant was carefully decanted and used for further analysis.

### Test Sample Preparation:

Two samples were analyzed in duplicates and the other two were prepared and analyzed with one replicate. Individual sample were prepared as follows: 1 mL aliquot of extract sample was transferred to a preconditioned 15 mL polypropylene (PP) centrifuge tube, to which 0.1 mL of nitric acid and IS were pipetted. Solutions were diluted to 5 mL with deionized (DI) water. The resulting test solutions were taken for ICP-MS/MS analysis, as described below

### Preparation of Pre-Dissolution Spikes (Fortified Samples);

For in-process verification of method accuracy, additional replicates of materials were fortified with known levels of target elements, and then otherwise prepared and analyzed in triplicate according to the procedures described above

### Calibration Curve Preparation;

Working standards were prepared by combining appropriately diluted reference standards (Inorganic Ventures and High Purity Standards, covering the concentration range of interest, together with selected internal standard. The final composition of these working standards was matched to the prepared samples for acid content. Results from ICP-MS/MS analysis of these working standards yielded the requisite data to prepare calibration curves for quantitation of each targeted element. 3

### ICP-MS/MS Analysis Conditions;

Prepared standards, test samples, and fortified test samples were analyzed using an Agilent 8800 ICP-MS/MS (triple quadrupole), employing various detection schemes optimized for each targeted element, and appropriate accessories. Instrumentation and detection schemes were optimized according to manufacturer recommendations.

TABLE 1
Peroxide Stability

| | | | | % H₂O₂ Remaining | % H₂O₂ Remaining |
|---|---|---|---|---|---|
| | | | | in Dentifrice | in Dentifrice |
| | Slurry | γ/β | Total Soluble | Composition After | Composition After |
| Abrasive Sample | pH | ratio | Metal (ppm) | 10 Days at 50° C. | 20 Days at 50° C. |
| Cal Pyro Ex. 1 | 5.95 | 1.54 | 101.19 | — | — |
| Cal Pyro Ex. 2 | 5.78 | 1.73 | 105.79 | — | — |
| Cal Pyro Ex. 3 | 5.77 | 1.46 | 103.81 | — | — |
| Cal Pyro Ex. 4 | 5.7 | 1.31 | 239.97 | 92.4 | 88.0 |
| Cal Pyro Ex. 5 | 5.47 | 1.06 | 314.32 | 91 | 87.1 |
| Cal Pyro Ex. 6 | 5.28 | 0.43 | 134.91 | 88.3 | 84.1 |
| Cal Pyro Ex. 7 | 5.23 | 1.04 | 134.91 | 89.1 | 87.4 |
| Cal Pyro Ex. 8 | 5.2 | 0.44 | 507.90 | 88.2 | 82.2 |
| Cal Pyro Ex. 9 | 4.72 | 0.09 | 1049.65 | — | 77.7 |

TABLE 1 shows the stability of peroxide using a variety of dentifrice compositions including different calcium pyrophosphate samples (Cal Pyro Ex. 1-9). The remaining components of the dentifrice composition are shown in TABLE 2. Slurry pH of the abrasive source alone are shown in TABLE 1. The gamma polymorph to beta polymorph (γ/β) ratio of the abrasive is also shown in TABLE 1. The total soluble metal content (Cr, Mn, Fe, Co, Ni, Cu, and Mo) is shown in TABLE 1.

TABLE 1 shows that calcium pyrophosphate samples with a slurry pH of at least about 5.4, a γ/β ratio of at least about 1, and/or a total soluble metal content of less than 600 ppm were more stable when combined in a dentifrice with hydrogen peroxide. For example, samples include a calcium pyrophosphate γ/β ratio of 1.06 (Cal Pyro Ex. 5) and 1.31 (Cal Pyro Ex. 4) led to over 90% remaining hydrogen peroxide after being stored for 10 days at 50° C.

TABLE 2
Compositions

| | Example 1 | Example 2 |
|---|---|---|
| Water | 56.63 | 54.31 |
| Glycerin | 10 | 10 |
| Amphiphilic Polymer¹ | 2.25 | 1.375 |
| Sodium Lauryl Sulfate (28%) | 5 | 7 |
| Alkyl Alcohol¹ | 1.25 | 1.25 |
| Sodium Monofluorophosphate | 1.14 | 1.14 |
| Phosphate Salts | 1.0 | 1.0 |
| Sucralose | 0.3 | 0.3 |
| Calcium Pyrophosphate | 10 | 10 |
| H₂O₂ (35%) | 11.43 | 11.43 |
| Flavor | 1.2 | 1.54 |
| Total | 100 | 100 |

¹Sepimax Zen ™
²Cetyl Alcohol:Stearyl Alcohol:SLS (45:45:10)

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention

What is claimed is:

1. An oral care composition comprising:
(a) peroxide;
(b) calcium pyrophosphate; and
(c) water,
wherein the oral care composition comprises a ratio of gamma phase calcium pyrophosphate to beta phase calcium pyrophosphate of at least about 0.8.

2. The oral care composition of claim 1, wherein the peroxide comprises hydrogen peroxide, urea peroxide, carbamide peroxide, calcium peroxide, sodium peroxide, zinc peroxide, sodium percarbonate, polyvinylpyrrolidone-hydrogen peroxide complex, cross-linked polyvinyl pyrrolidone-hydrogen peroxide complex, or combinations thereof.

3. The oral care composition of claim 1, wherein the oral care composition comprises at least about 1%, by weight of the oral care composition, of the peroxide.

4. The oral care composition of claim 1, wherein the oral care composition comprises at least about 3%, by weight of the oral care composition, of the peroxide.

5. The oral care composition of claim 1, wherein the oral care composition comprises at least about 3.5%, by weight of the oral care composition, of the peroxide.

6. The oral care composition of claim 1, wherein the oral care composition comprises from about 5%, to about 75%, by weight of the oral care composition, of the water.

7. The oral care composition of claim 1, wherein the oral care composition comprises from about 1%, to about 30%, by weight of the oral care composition, of the calcium pyrophosphate.

8. The oral care composition of claim 1, wherein the oral care composition comprises from about 1% to less than 15%, by weight of the oral care composition, of the calcium pyrophosphate.

9. The oral care composition of claim 1, wherein the ratio of gamma phase calcium pyrophosphate to beta phase calcium pyrophosphate is at least about 1.

10. The oral care composition of claim 1, wherein the oral care composition is a dentifrice composition.

11. The oral care composition of claim 1, wherein the oral care composition comprises fluoride.

12. The oral care composition of claim 11, wherein the fluoride comprises sodium fluoride, stannous fluoride, sodium monofluorophosphate, amine fluoride, or combinations thereof.

13. The oral care composition of claim 11, wherein the fluoride comprises sodium monofluorophosphate.

14. The oral care composition of claim 1, wherein the oral care composition comprises metal.

15. The oral care composition of claim 14, wherein the metal comprises tin, zinc, copper, or combinations thereof.

16. The oral care composition of claim 15, wherein the tin comprises stannous chloride, stannous fluoride, or combinations thereof.

17. The oral care composition of claim 15, wherein the zinc comprises zinc lactate, zinc oxide, zinc phosphate, zinc citrate, or combinations thereof.

18. The oral care composition of claim 1, wherein the oral care composition comprises amino acid.

19. The oral care composition of claim 18, wherein the amino acid comprises arginine, histidine, lysine, aspartic acid, glutamic acid, serine, threonine, asparagine, glutamine, cysteine, selenocysteine, glycine, proline, alanine, valine, isoleucine, leucine, methionine, phenylalanine, tyrosine, tryptophan, citrulline, ornithine, creatine, diaminobutanoic acid, diaminoproprionic acid, salts thereof, or combinations thereof.

20. The oral care composition of claim 1, wherein the oral care composition comprises anionic surfactant.

21. The oral care composition of claim 20, wherein the oral care composition comprises at least about 1.5%, by weight of the oral care composition, of anionic surfactant.

22. The oral care composition of claim 20, wherein the oral care composition comprises greater than 1.5%, by weight of the oral care composition of anionic surfactant.

23. The oral care composition of claim 22, wherein the anionic surfactant comprises sodium alkyl sulfate surfactant.

24. The oral care composition of claim 23, wherein the sodium alkyl sulfate surfactant comprises sodium lauryl sulfate.

25. The oral care composition of claim 1, wherein the oral care composition comprises amphiphilic polymer.

26. The oral care composition of claim 25, wherein the amphiphilic polymer does not form an isolatable complex with the peroxide.

27. The oral care composition of claim 25, wherein the amphiphilic polymer comprises a macromolecule with a hydrophobic portion and a hydrophilic backbone.

28. The oral care composition of claim 25, wherein the amphiphilic polymer comprises a 2-acrylamido-2-methylpropane sulfonic acid polymer, copolymer, cross polymer, or combination thereof.

29. The oral care composition of claim 1, wherein the oral care composition has a slurry pH of from about 4 to about 8.

30. The oral care composition of claim 29, wherein the slurry pH is from about 4 to about 7.

31. The oral care composition of claim 1, wherein the oral care composition comprises alkyl alcohol.

32. The oral care composition of claim 31, wherein the alkyl alcohol comprises cetyl alcohol, stearyl alcohol, or combinations thereof.

33. The oral care composition of claim 1, wherein the oral care composition has at least about 90% of peroxide remaining after the oral care composition has been stored at 50 ° C. for 10 days.

34. An oral care composition comprising:
(a) peroxide;
(b) abrasive; and
(c) water,
wherein the abrasive comprises less than about 0.0006%, by weight of the oral care composition, of soluble metal selected from the group consisting of Cr, Mn, Fe, Co, Ni, Cu, and combinations thereof.
